(19) United States
(12) Patent Application Publication
(10) Pub. No.: US 2022/0010368 A1
(43) Pub. Date: Jan. 13, 2022

(54) ENHANCED DETECTION OF LOW-COPY-NUMBER NUCLEIC ACIDS IN AN INTEGRATED WORKFLOW

(71) Applicant: Biocartis NV, Mechelen (BE)

(72) Inventor: Max Haesendonckx, Mechelen (BE)

(21) Appl. No.: 17/294,982

(22) PCT Filed: Nov. 18, 2019
(86) PCT No.: PCT/EP2019/081683

§ 371 Date: May 18, 2021

(30) Foreign Application Priority Data
Nov. 19, 2018 (EP) 18207092.0

(51) Int. Cl. C12Q 1/6848; C12Q 1/6806

(52) U.S. Cl. CPC C12Q 1/6848; C12Q 1/6806

(57) ABSTRACT

The present invention concerns methods and strategies of detecting low-copy-number nucleic acids in integrated work-flows on automated or semi-automated platforms, using a pre-amplification reaction positioned to only a portion of a silica surface in an extraction chamber of the platform. The present methods of the invention open the possibility to expand the repertoire of already developed automated workflows to enable them to process even very diluted samples such as bodily fluids, including liquid biopsies.

### FIELD OF THE INVENTION

The present invention concerns methods and strategies of detecting low-copy-number nucleic acids in integrated workflows on automated or semi-automated platforms. The present methods of the invention open the possibility to expand the repertoire of already developed automated workflows to enable them to process even very diluted samples such as bodily fluids, including liquid biopsies.

### BACKGROUND TO THE INVENTION

In monitoring of physiological and/or pathological states in patients, liquid biopsy research has gained a lot of attention. This is mainly because it offers non-invasive sampling potentially allowing early diagnosis and frequent monitoring. A good example of this is detection of circulating tumour markers (ctDNA) in body fluids during early stages of cancer. However, due to their loads simply being very low per sample volume, their effective detection is extremely challenging and requires not only highly sensitive and robust diagnostic assays but also the ability to process large sample volumes.

There currently exist several excellent highly sensitive platforms offering robust genetic marker testing, using diagnostic fluidic cartridges. One of the advantages such systems is their compact handheld design, which greatly facilitates ease of use and storage considerations. Given that dimensional minimization is a general trend in point of care (POC) devices, and that many of them make use of a silica-based Boom extraction that requires large reagents volumes including e.g. chaotropic buffer, the common problem these devices face is the limited room for accepting the necessary sample volumes in order to be suitable for detecting low copy number nucleic acids.

Consequently, even despite the constant development of more sensitive chemistries and detection techniques, it remains extremely challenging to develop a robust liquid biopsy assay based on currently existing integrated workflows and using handheld devices without having to change the platform design and/or without disrupting the ease of use and compactness of the currently available assays. This is because the detection of low-copy-number nucleic acids in standard automated and semi-automated fluidic workflows appears to require higher than usual sample inputs and keeping to a minimum material losses stemming from inherent to a given device extraction efficiencies and sample distribution considerations.

To address these issues we have developed an integrated approach of:

(i) specially-localized, preferably by pressure, nucleic acid pre-amplification protocol in DNA extraction place in the presence of silica solid support, even a one that fills in the volume of the extraction chamber, prior to the transferring of the silica-extracted nucleic acids downstream of integrated workflows, which inevitably generates dead volumes and therefore also losses or dilutions of the precious low-copy number nucleic acids; preferably preceded by

(ii) using a solid phase pre-capture technology based on at least on ionic exchange and preferably also affinity, preferably being a hydroxyapatite (HAP)-based nucleic acid pre-capture technology.

Furthermore, concerning (i), as majority of integrated diagnostic devices require the purified nucleic acid sample to be distributed over multiple reaction chambers, it should be noted that when dealing with very low loads of e.g. ctDNA, said distributing even further lowers the probability of detecting the low copy nucleic acid target of choice and, hence the sensitivity of the assay. Pre-amplification, defined as amplification of extracted nucleic acids prior to their distribution to another amplification chamber or chambers where usually the detection takes place, provides an exponential increase of the targeted DNA. This is highly desirable for low-copy number targets as it allows increasing the sensitivity of the downstream analyses by eliminating the dilution factor and steering the sample away from Poisson distribution statistics. It also provides simplification of the sample, with targets being amplified relative to the non-amplified genomic material. This may substantially reduce selectivity pressures of the downstream assay by reducing the likelihood of nonspecific amplification and its side effects.

As the above advantages are recognized in the field, targeted pre-amplification of low copy number nucleic acids prior to downstream analysis, such as NGS and qPCR, is generally known in bench-top protocols and performed after the nucleic acid extraction and purification. It follows from the latter that in the existing art, there is a strict spatio-temporal distinction between the extraction and purification process and the (pre-)amplification process. The same applies to the automated workflow cartridge-based systems. For example, the FilmArray system of BioFire (BioMérieux) is designed to provide for pre-amplification of bench-top purified and extracted DNA, and then to distribute the pre-amplified genetic material into many different PCR chambers. To our knowledge, the existing automated or semi-automated (i.e. partially bench-top) workflows, overcome the dilution issue by either accepting the bench-top pre-amplified material or by being equipped with an additional pre-amplification chamber already from the beginning of the system's design, just like in the example mentioned above. The majority of automated or semi-automated fluidic POC devices, however, do not comprise such dedicated upstream pre-amplification space and would frequently need to be redesigned or rebuilt to acquire the upstream pre-amplification functionality. However, the redesigning of an existing system may take several years of testing and a substantial investment that in practice many molecular diagnostics companies will not commit to. This is because when working with semi-or fully automated molecular diagnostics systems, implementation of a pre-amplification protocol is far from straightforward. When the molecular analysis is performed in a disposable cartridge, limited opportunities exist to find place for or integrate an additional chamber for an upstream pre-amplification reaction. Many of such cartridges, however, already contain an extraction chamber filled with a silica solid support and frequently also having some temperature-control functionalities. Nucleic acid thermocycling in the presence of such silica filling is known however to be inefficient, suffer from inhibition, and yield a broad range of non-specific products. We have overcome these problems by providing a volume-localized, in particular by use of pressure, PCR amplification protocol of silica-captured DNA in the extraction chamber, which allowed us to detect low copy number targets in an integrated cartridge without the need of modifying the cartridge's design.

We observed that for large volume and/or diluted liquid samples, like urine, the effect was further enhanced by preceding the step of pre-amplification on silica with a pre-capture step to a hydroxyapatite (HAP) solid support. Although HAP appears to be the most suitable pre-capture technology, in theory other ionic exchange and preferably also affinity-based pre-capture strategies could be suitable for integration in the presented herein integrated workflows. One potential promising candidate is GEAE Sephacel. Other technologies could also be envisaged, provided they meet the following considerations. First, the amount of buffers, such as equilibration buffers, washing buffers, elution buffers etc., and their required volumes should be as limited as possible, due to the fact a buffer storage space is a major challenge when upscaling sample volume in integrated system, especially the ones using cartridges. Third, the DNA extraction efficiency of the pre-capture technology should be close to 100%, especially if short ctDNA is the preferred targets. Lastly, the elution volume should be as low as possible as this will require less chaotropic binding buffer for further Boom extraction. And finally, the eluted product should be compatible with Boom extraction technology, thus not impacting the silica-DNA binding mechanism.

As there exists a high need for nucleic acid isolation mechanisms that are able to handle large sample volumes (i.e. ≥10 ml), and require little or no additional binding agents, we have created a rapid hydroxyapatite-based nucleic acid-specific pre-capture protocol that meets the above-specified criteria. Consequently, the presented herein HAP-based strategy is compatible with integrated workflows and can easily be introduced to the sample-receiving section of handheld POC devices. In particular, the protocol not only provides efficient isolation of DNA from 10 mL or more of biological fluid, like plasma, resulting in n-fold volume reduction, but also at the same time results in sample pre-clearing and simplification by removing the bulk of proteins.

HAP-based nucleic acid extraction is a solid-phase extraction (SPE) method using both ionic exchange and affinity principles. Methodologically it resembles anion-exchange chromatography. Multiple designs of HAP-based DNA extraction have been previously described in scientific literature, targeting a variety of sample types. Several of them include:
S. Yu et al./J. Chromatogr. A 1183 (2008) 29-3;
J. Mater. Chem. B, 2014, 2, 6953-6966;
Colman, M. J. Byers, S. B. Primrose, and A. Lyons: Rapid Purification of Plasmid DNAs by Hydroxyapatite Chromatography;
P. Gagnon, P. Ng, J. Zhen, C. Aberin, J. He, H. Mekosh, L. Cummings, S. Zaidi, R. Richieri, A ceramic hydroxyapatite based purification platform;
Purdy K J, Embley T M, Takii S, Nedwell D B. Rapid Extraction of DNA and rRNA from Sediments by a Novel Hydroxyapatite Spin-Column Method. Applied and Environmental Microbiology. 1996;62(10):3905-3907.
   These SPE and other known to us HAP-based methods elute DNA in a high -salt or -phosphate environment, rendering a product that is incompatible with further downstream molecular analysis.

The hydroxyapatite-based isolation of nucleic acids from biological samples is currently mostly being done via a liquid chromatography column matrix or using a spin-column. Most approaches enable the binding of nucleic acids to HAP by providing a threshold concentration of potassium- or sodium phosphate, or other salts, at a neutral pH (typically around pH 7.0). By further increasing the concentration of phosphate ions (up to 500 nM), DNA is eluted from the HAP matrix. While 500 nM of phosphate provides optimal elution conditions, such high concentrations are not compatible with further downstream analysis due to their inhibitory effect. Frequently, a compromise is being made between elution efficiency and the performance of the subsequent DNA analysis by lowering the phosphate concentration during elution.

Possibly, because of this high-salt content in the eluted product, HAP-based enrichment is not used in integrated workflows. To our knowledge, at least no integrated PCR-analysis-based workflow or cartridge has been developed to date, likely because phosphate salts are known to exert inhibitory effects on PCR. In the settings of the present invention, we confirmed however, that a silica-based extraction of the HAP-enriched nucleic acids followed by pressure-controlled pre-amplification protocol on the silica solid support, yields robust improvement in detection of low copy number nucleic acid targets in a fully automated POC device. Demonstration of this result and other advantages of the present invention are presented in continuation.

### SUMMARY OF THE INVENTION

The present invention is defined in the appended independent claims. Preferred embodiments are defined in the dependent claims. In particular, the present invention concerns a method of detecting a low copy number nucleic acid in an automated system, said system comprising an extraction chamber being at least partially filled a silica solid support, and an amplification chamber, the method comprising:
providing a sample comprising a low copy number nucleic acid to an extraction chamber of the automated system, said extraction chamber comprising silica surface for DNA adsorption and being adjacent to at a heater;
adsorbing the nucleic acid to the silica surface;
performing pre-amplification of the nucleic acid in the presence of the silica surface;
eluting the pre-amplified nucleic acid from the silica surface;
transporting the pre-amplified nucleic to an amplification chamber of the automated system, said amplification chamber being fluidly connected with the extraction chamber;
amplifying the pre-amplified nucleic acid in said amplification chamber; wherein the automated system is configured
to position the pre-amplification reaction to only a portion of the silica surface occupying the zone of the extraction chamber adjacent to the heater, and
to maintain substantially free of the pre-amplification reaction the remaining portion of the silica surface within the extraction chamber.

In a preferred embodiment, the positioning is done by pressure control. In particular, the pre-amplification is performed under pressure control configured to position the pre-amplification reaction to the portion of the silica positioned in the zone of the extraction chamber adjacent to the heater, such that another zone of the extraction chamber not being adjacent to said heater and filled with a different portion of the silica is kept void of the pre-amplification reaction. Most commonly, the inner volume of the extraction chamber will be at least partially filled, possibly entirely filled, with silica matrix that provides the silica surface for the nucleic acid extraction. Possibly said silica matrix will be provided as a membrane or as a block of any geometrical shape, possibly corresponding to the shape of inner space of the extraction chamber. Silica membranes are well known in the art. The blocks can be made of siliceous fibers or beads having different levels of integration. The blocks may fill the inner volume of the extraction chamber entirely or partially. In the latter case, the silica matrix can be provided as a block of any preformed geometrical shape that is fitted into the inner space of the extraction chamber such that is remains therein. Examples of such blocks could be e.g. layered structure made of stacked silica sheets. Different designs of silica extraction surfaces are known and commonly include resins, beads, parallelized strictures like layers of sheets. Their temperature conducing properties can vary depending on the density, porosity, and/or spaces between particles as well as their shapes and structure. Porous silica is in general a very poor temperature conductor and thermocycling conditions of pre-amplification should be carefully fine-tuned in every system to avoid generation of aspecific products. The material and thickness of the wall of the amplification chamber will also have an influence. In general, taking into account the above considerations, the positioning of the pre-amplification reaction in the silica solid support within the pre-amplification chamber from the heater should not extend beyond 7, 6, or 5 mm, preferably not beyond 4 mm, and most preferably should be confined to not extend beyond 3 mm from the heater.

In another preferred embodiment, the pre-amplification comprises symmetrical heat cycling between a top and a bottom temperature values, which provides a temperature profile that is particularly advantageous for cycling temperatures for nucleic acid amplification in a thick block of silica.

In a particularly preferred embodiment of the previous embodiment, each of the top and the bottom temperature are held for at least 30 seconds, preferably for at least 45 second, most preferably for at least 1 minute.

In another preferred embodiment, the pre-amplification in performed in presence of a silica blocking component, preferably being bovine serum albumin (BSA). In a particular embodiment, the blocking component, preferably being BSA, is provided in a pre-amplification buffer. In a preferred embodiment of the latter, the concentration of BSA in the amplification buffer is comprised between 0.1 and 5 ug/ul, preferably between 0.2 and 4 ug/ul, more preferably between 0.5 and 3 ug/ul, and most preferably between 1 and 2 ug/ul.

In a most preferred embodiment, the method of the invention is provided, wherein the sample comprising the low copy number nucleic acid was obtained by contacting a biological sample with a hydroxyapatite (HAP) solid support.

In another aspect, as we have observed that HAP pre-treatment alone also provides a substantial advantage to detecting low copy number nucleic acids in automated workflows and that it is generally advantageous for simplifying the biological sample prior to its processing in an integrated workflow, the present invention also provides a general HAP-based method of detecting a low copy number nucleic acid in an automated system, the method comprising:
contacting a biological sample with a hydroxyapatite (HAP) solid support to obtain a sample comprising a low copy number nucleic acid;
providing the sample comprising the low copy number nucleic acid to an extraction chamber of the automated system, said extraction chamber comprising silica surface for DNA adsorption;
adsorbing the nucleic acid to the silica surface; and
amplifying the nucleic acid within the automated system.

In the above embodiment, the sample comprising the low copy number nucleic acid is a result of eluting the nucleic acid captured to the HAP solid support. This eluting is preferably done in a phosphate buffer preferably comprising KHPO4, which is covered in in more detail in continuation. For optimal result however, in most preferred embodiments of the above embodiment, the step of amplifying is preceded by pre-amplifying of the nucleic acid adsorbed to the silica surface, and optionally by eluting and transporting the pre-amplified nucleic acid to an amplification chamber of the automated system having the amplification chamber fluidly connected with the extraction chamber.

In a preferred embodiment of any of the above, the contacting of the biological sample with the HAP solid support is performed in the presence of monovalent or bivalent cations that enhance binding of nucleic acids to HAP. In preferred embodiments, the contacting of the biological sample with the HAP solid support is performed in the presence of Na+, Li+, or Mg2+ cations. In preferred embodiments, the concentration of the cations is comprised between 0.1 M and 2 M. In most preferred embodiments, the contacting is performed in the presence of Na+ or Li+ cations at a concentration above 0.5 M, preferably above 0.75 M, most preferably above 1 M, and preferably also being below 3 M, more preferably below 2.5 M, most preferably being not more than 2 M. In an alternative embodiment the contacting is performed in the presence of Mg2+ cations at a concentration not exceeding 1 M, preferably below 0.75 M, most preferably below 1 M, and preferably also being above 30 nM, more preferably being equal or above 45 nM, most preferably being equal or above 50 nM. In a particular embodiment, the contacting of the biological sample with the HAP solid support is performed in the presence of Na+ cations at a concentration of about 1 M and/or in the presence of Mg2+ cations at a concentration of about 100 mM.

In yet another embodiment of the method of the invention, the sample comprising the low copy number nucleic acid is a result of eluting the nucleic acid captured to the HAP solid support with a phosphate buffer. In a preferred embodiment of the latter, the pH of the buffer is between 6.2 and 7.4, preferably between 6.4 and 7.2, more preferably between 6.6 and 7, and most preferably is around 6.8. In another preferred embodiment, the phosphate buffer comprises KHPO₄. In a preferred embodiment, the concentration of KHPO₄ in the phosphate buffer is between 110 mM and 500 mM, wherein the range between 130 mM and 170 mM allows for preferential binding of short DNA, i.e having a length range between 100 and 500 bp and mostly being <200 bp. In a preferred embodiment, the eluting is performed at HAP concentration between 0.2 and 0.5 M, preferably at about 0.5 M, being a concentration which is assumed to elute all DNA strands from HAP.

In a preferred embodiment, the methods of the invention are performed on a biological sample that is a bodily fluid. In a preferred embodiment, the bodily fluid is selected from plasma, serum, blood, urine, CSF, bile, saliva etc., and preferably is a mammalian, more preferably a human bodily fluid. However, it should be noted that the methods of the invention can be applied on all possible liquid samples including tissue lysates or cell suspensions in e.g. culture media or PBS etc.

In a final aspect, the present invention also relates to automated systems, workflows, and/or cartridges adapted to performing any of the methods of the invention.

### BRIEF DESCRIPTION OF THE DRAWINGS

For a fuller understanding of the nature of the present invention, reference is made to the following detailed description taken in conjunction with the accompanying drawings in which:

FIG. 1: illustrates the temperature gradient simulation in silica-based extraction chamber across the extraction membrane during heat cycling;

FIG. 2: shows an example of pressure-based dosing of a specific volume of a PCR buffer onto a silica solid support;

FIG. 3: shows a schematic overview of the pre-amplification and qPCR detection workflow.

FIG. 4: shows variation in pre-amplification efficiencies over time. Each plot represents variation of qPCR Cr-values in a set of sample repeats in a single experiment;

FIG. 5: shows improved robustness of automated pre-amplification on silica solid support when dosing accuracy is increased due to the pressure-based approach;

FIG. 6: shows binding conditions of cell-free DNA (cfDNA) to HAP matrix in function of cation types and their concentrations, the DNA amounts are expressed as OD260 measurements of the unbound cfDNA fraction in the supernatants of the HAP-extracted plasma samples. Low detection of DNA represents a more efficient binding;

FIG. 7: shows binding efficiency of cfDNA to HAP matrix at different concentrations of Na⁺ or Mg²⁺; the readout is expressed as qPCR Ct-values obtained for a house-keeping HPRT1 gene;

FIG. 8: zoom in on cfDNA binding efficiency to HAP matrix at Mg²⁺ concentrations lower than shown in FIG. 7; the readout is expressed as qPCR Ct-values obtained for a house-keeping HPRT1 gene;

FIG. 9: shows binding efficiency of cfDNA to HAP matrix at different concentrations of K⁺ or NH₄⁺; the readout is expressed as qPCR Ct-values obtained for a house-keeping HPRT1 gene;

FIG. 10: shows correlation between DNA fragment size and elution efficiency from HAP matrix in different concentrations of KHPO₄ phosphate buffer;

FIG. 11: shows efficiency of the centrifugation-based HAP-extraction protocol performed on cfDNA from a 10 mL plasma and processed using Idylla cartridge with silica-based extraction chamber side by side with a regular non HAP-enriched 1 mL plasma sample. The eluted products were analysed by qPCR;

FIG. 12: shows the potential of the complete sequential workflow, including HAP-based pre-capture of cfDNA from 10 mL of plasma followed by silica-based extraction and pre-amplification of the silica-captured DNA in an automated system (Biocartis Idylla).

### DETAILED DESCRIPTION OF THE INVENTION

When working with semi- or fully automated molecular diagnostics systems, implementation of a pre-amplification protocol can be difficult as the system needs to be equipped with a suitable reaction compartment. As part of the present invention, we have circumvented this need by creating a method that allows a robust amplification of nucleic acids directly in their silica-captured form within nucleic acid extraction/purification chambers. For clarity, said amplification on silica-solid support in the will be further referred to herein as “pre-amplification” due to the fact that most of the existing systems use DNA amplification to detect the nucleic acid target in the final stages of performing an assay.

The present method relies on pressure-based volume control of the pre-amplification reaction mixture that positions it in such vicinity from the heater that the thermocycling profiles become sufficiently specific to balance out poor thermal conductivity of silica and result is robust PCR. Depending on the extraction chamber's properties and dimensions, the pre-amplification efficiency and specificity of the methods of the invention can further be improved by performing symmetric heat cycling on the silica surface and by tweaking the contents of the PCR buffer. The presented herein method has the potential to be integrated into any fully automated system comprising a silica-based extraction chamber for nucleic acid processing.

The advantages of the presented herein solution include removal of stochastic effects affecting detection of low-copy number target nucleic acids in established automated and semi-automated workflows by enabling a pre-amplification protocol in a disposable cartridge with fixed configuration in which no distinct location is foreseen for pre-amplification. Present methods provide a fully integrated approach that does not require specialized equipment or infrastructure, which because of its integrated nature additionally reduces the chance of amplicon contamination. In contrast to existing systems that contain a dedicated pre-amplification compartment, a further advantage of our approach is that the pre-amplification is done in the extraction chamber directly on the solid extraction support, thereby further minimizing the possibility of losing material during downstream transfer and/or because of dead volumes.

The proof of principle approach of the method of the invention can be demonstrated using Idylla™ cartridge belonging to Biocartis NV, but, as it will be apparent to any skilled person, it can be applied to any commercial integrated system comprising a nucleic acid extraction with silica. In present example, human plasma was analysed in a fully automated manner in a proprietary to Biocartis NV BRAF mutation-detection cartridge whose extraction chamber is packed with multiple silica sheets and closed by syringes at both the entrance and the exit. During the sample extraction, DNA selectively binds to the silica membrane, while contaminants are washed away. The binding buffer used contains 3.68M GuSCN and 43% ButOH, while the washing steps of the silica sheets was performed with 90% ethanol. After the washing, the membrane was dried with hot air. Subsequently, a specific volume of PCR buffer was accurately dosed in an automated manner onto the silica membrane by use of the syringes. For storage reasons, the PCR buffer components can be provided in a spotted and dried or lyophilized form within the cartridge but can also be provided in a solution. The pressure-based approach allows the PCR buffer to be accurately dosed into the silica and to make it pass through and address all the silica-captured DNA, while later allowing to limit the reaction volume to a specific area of the extraction chamber where the temperature cycling profiles are most promising.

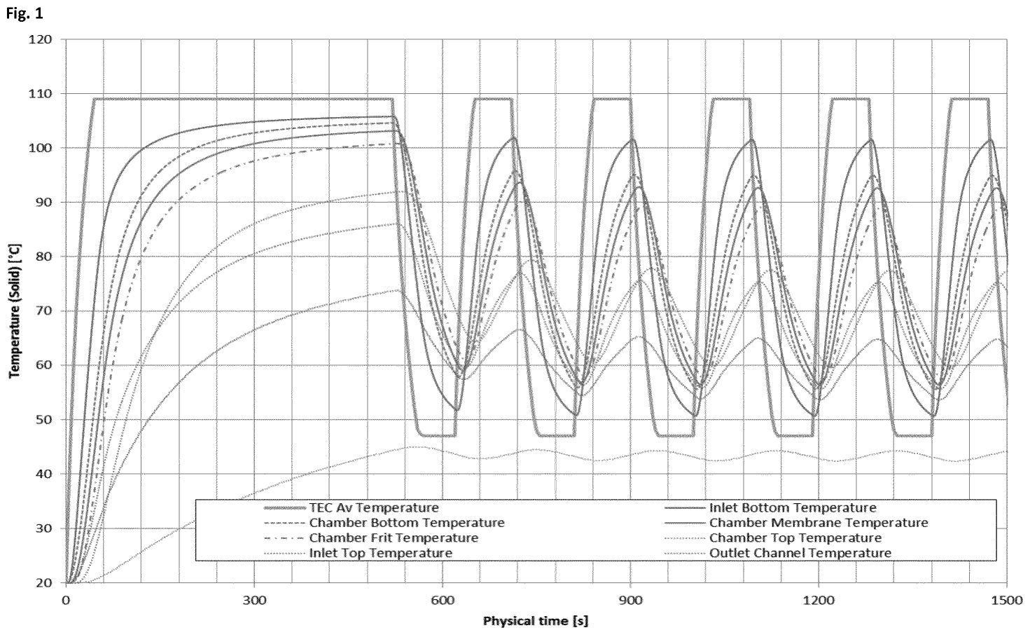

In the proof-of-concept example, the extraction chamber is docked into an aluminium cup. The temperature of the cup is regulated by a Peltier element. We found that symmetrical heat cycling of the cup was the most appropriate strategy to be applied to the particular extraction chamber design in order to allow the pre-amplification of the targeted DNA in a robust manner. FIG. 1 illustrates temperature gradients across different zones of the extraction membrane during an exemplary heat cycling. The uppermost continuous grey line represents the temperature of the extraction cup which acts as the heat source as controlled by a Peltier element. From the figure it becomes apparent that only a limited area of the extraction chamber allows for temperature change profiles that are suitable for functional DNA thermocycling.

Typically, top- and bottom temperatures are held at their set point for at least 1 minute, for a limited amount of cycles (13). The temperature cycling profile was as follows:

### Hotstart: 300″ 109° C.

+13 cycles of;

### Denaturation: 60″ at 109° C.

### Annealing: 60″ at 47° C.

The amplified DNA can be recuperated by flushing at room temperature the extraction chamber with any low ionic strength solution such as water or PCR buffer, etc.

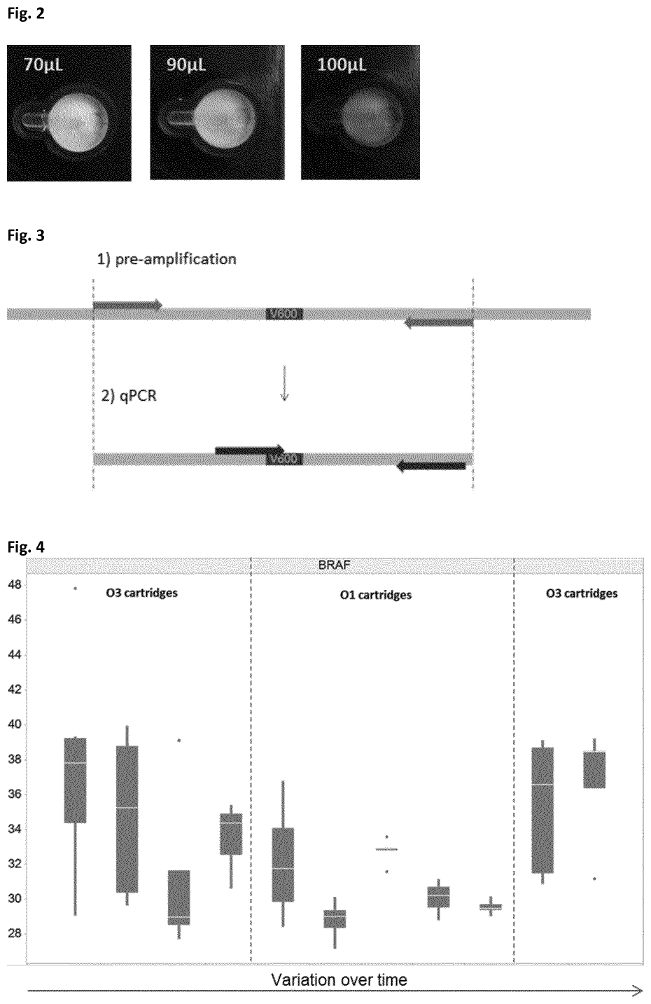

FIG. 2 shows an example of accurate dosing of a specific volume of PCR buffer onto the silica membrane. The photography shows the extraction chamber of the cartridge dosed with different volumes of a dextran blue solution. The dosage is automated and pressure-based. In this particular cartridge model, a volume of 90 μL, dosed from a back end manifold of the cartridge addresses all captured DNA while at the same time limiting the reaction volume to a specific area of the extraction chamber. Naturally, in different cartridge models, different volumes of dosing may have to be estimated for obtaining optimal results, as it will be appreciated by and is within the scope of abilities of any skilled person.

Next, the silica-extracted and pre-amplified DNA is then eluted towards the mixing chamber of the cartridge in a total volume of 250 μL. 1/10th of this mixture is then addressed in the downstream qPCR assay, targeting the BRAF gene. FIG. 3 shows the schematic overview of the target BRAF V600 mutation assay comprising the pre-amplification step according to the invention. Arrows symbolize the primers used in both of the pre-amplification and the qPCR steps. The outer amplicon is pre-amplified for 13 cycles in the cartridge extraction chamber, as described above. The inner qPCR amplicon is amplified and detected using the following reagents and conditions:

### Hotstart: 5′ 95° C.

+50 cycles of;

### Denaturation: 5″ at 95° C.

### Annealing: 2″ at 65.5° C.

19″ at 64° C.
   PCR buffer composition:

### 50 mM KCl 10 mM Tris pH 8.6

### 3 mM MgCl2

0.2 mM dNTP mix

### 0.2 U/μL Faststart

500 nM primers
250 nM probe
For more details, including primer sequences can be found in Bisschop et al. Melanoma Research 2018; 28(2):96-104.

FIG. 4 illustrates the variation in pre-amplification efficiency over time. Each plot represents a set of sample repeats in a single experiment. The Ct-values of the downstream qPCR assay are shown. These results were obtained based on inaccurate dosing of the PCR buffer onto the silica membrane. This resulted in variable preamp efficiency.

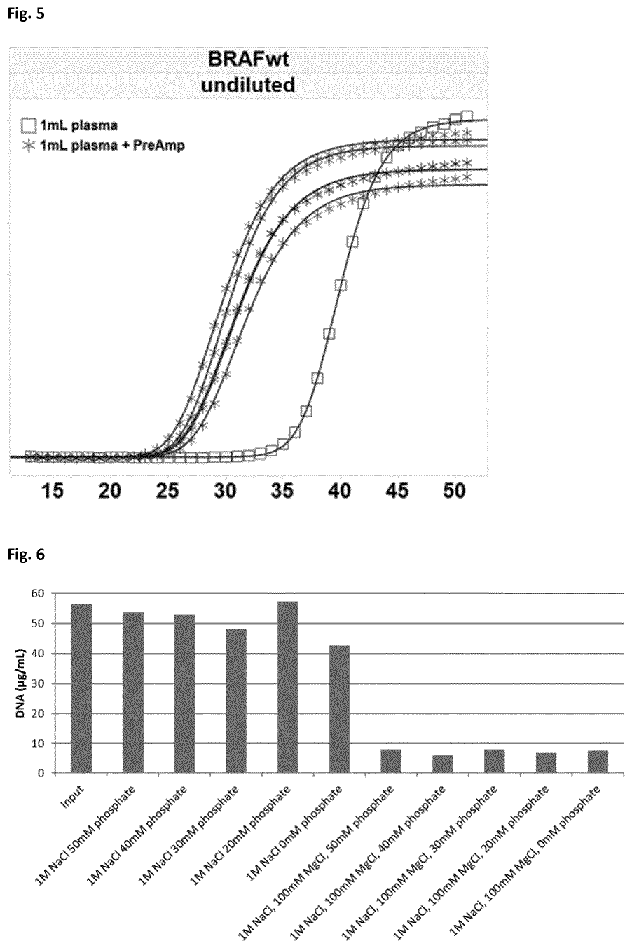

In contrast to the above, FIG. 5 shows the improved robustness of the workflow including the pre-amplification, when dosing accuracy is increased due to the pressure-based approach. The fluorescent readout of the downstream qPCR is visualised. Only 1/10^{th} of the preamplified product is addressed in the downstream assay. The asterisk-marked curves represent samples where a pre-amplification was implemented into the workflow. The square-marked signal curve represents a similar workflow without the pre-amplifications. 13 cycles of pre-amplification result in a highly satisfactory Ct-shift of 10, which is frequently more than enough to allow detection of low copy number targets that otherwise would be missed.

Many integrated platforms make use of a silica-based extraction method. As mentioned before, however, because the Boom extraction methodology requires large volumes of buffer and because POC devices need to be compact of preferably handheld dimensions, most automated systems only accept limited the sample volume. Consequently, there is a high need for nucleic acid isolation mechanisms that are able to handle large sample volumes, and require little or no additional binding agents. To supplement the above described pre-amplification methodology, we have additionally designed a rapid hydroxyapatite-based DNA pre-capture protocol that provides efficient isolation of DNA from large volumes of plasma (>10 mL). Our methodology, depending on the need and the type of the given system, can be implemented as part of a semi-automated workflow, as a supplemental bench-top centrifugation-based procedure, or as part of a fully automated workflow directly inside of a cartridge or via coupling a specific-volume-accommodating module to a cartridge.

The developed by us HAP method further enhances the low copy number target detection in the pre-amplification-based methods of the invention as well as in general. The present HAP-based DNA isolation method does not require addition of a buffer or anything other than solid salt and HAP in any form. Most commercial batches of HAP can be used, so the method is generic. Because of its simplicity, the method has the potential of being performed on multiple different biological fluids. The latter may even contain intact cells and depending on cation types and concentrations used during the incubation with HAP, the method gives the potential to only enrich the cell-free DNA (cfDNA) fraction without binding cells and thus the nucleic acids contained therein The nucleic acid elution method is optimized for maximum compatibility with downstream silica extraction. The proof-of-concept DNA elution is performed at a high (between 0.2 and 0.5 M) potassium phosphate salt concentration, ensuring the highest elution efficiency of all DNA bound to calcium groups of the HAP solid support. The negative impact of these high phosphate concentrations on the downstream PCR is countered by performing a subsequent silica-based clean up. Thanks to this combined approach, no compromises have to be made and both of the HAP-extraction and pre-amplification on silica can be performed as part of one continuous integrated workflow.

The HAP-based pre-treatment method comprises two steps being nucleic acid binding and elution, and proceeds to completion within minutes or less without the use of excessive amounts of HAP. A typical HAP commercial suspension may be added to no more than 1/20^{th} the volume. The conditions can be optimized to high specificity for DNA and nucleosomes. Due to the addition of a specific concentration of Mg²⁺ and Na⁺ counter ions, there is hardly any binding of plasma proteins. As witnessed by OD spectra from plasma eluates resembling OD spectra of pure DNA, with a maximum around 260 nm and very little absorption around 280 nm that indicative of tryptophan absorption of proteins. Use of specific concentrations of Mg²⁺ as a counterion allow to maximize binding to HAP of short DNAs (<200 bp) and even nucleosomes without the use of disruptive additives or heating. The eluted product consists of relatively clean nucleic acids, such as cfDNA, dissolved in phosphate buffer, which is compatible with most commercial downstream sample purification methods and easier to process than protein- and/or cell debris-containing lysates on fluidic or micro-fluidic platforms due to having much lower propensity to precipitation.

Most commercially available cfDNA extraction kits (i.e. QIAamp by QIAGEN) are silica-based and require a time consuming and expensive proteinase K digestion step in order to remove the bulk of proteins from the biological sample. The incorporation of a hydroxyapatite-based pre-capture provides sample simplification, by removing said proteins, prior to silica extraction and making the proteinase K digestion redundant. The pre-capture technology also provides n-fold reduction of the sample volume, which enables compatibility with the downstream silica extraction in handheld devices by decreasing the required chaotropic buffer volume.

As a first step, we have designed a centrifugation-based bench protocol that ensures fast and ˜100% efficient extraction of cfDNA from a 10 mL plasma sample. Firstly, 20 μL of a typical commercial HAP suspension (i.e. buffered aqueous suspension, 25.7% total solids) is added to 10 mL of plasma in a 50 mL falcon tube. Due to the significant reaction surface and binding capacity of the HAP matrix, inter- and intra-batch variation of the HAP suspension is trivial. The sample volume can be easily scaled up or down. Afterwards or immediately in the HAP and sample mixture, the appropriate salts are included, which is necessary to enhance the affinity of HAP to bind nucleic acids over its high affinity to bind proteins.

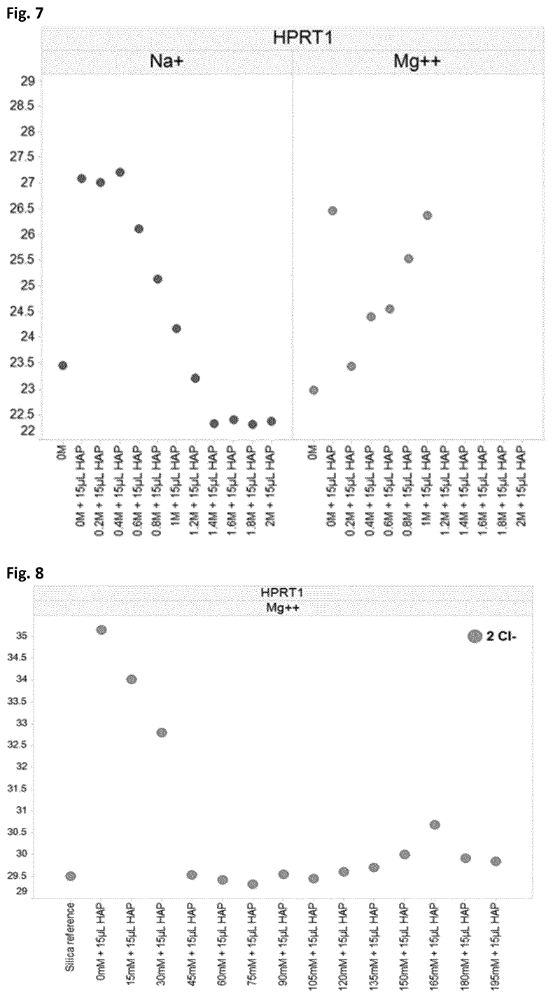

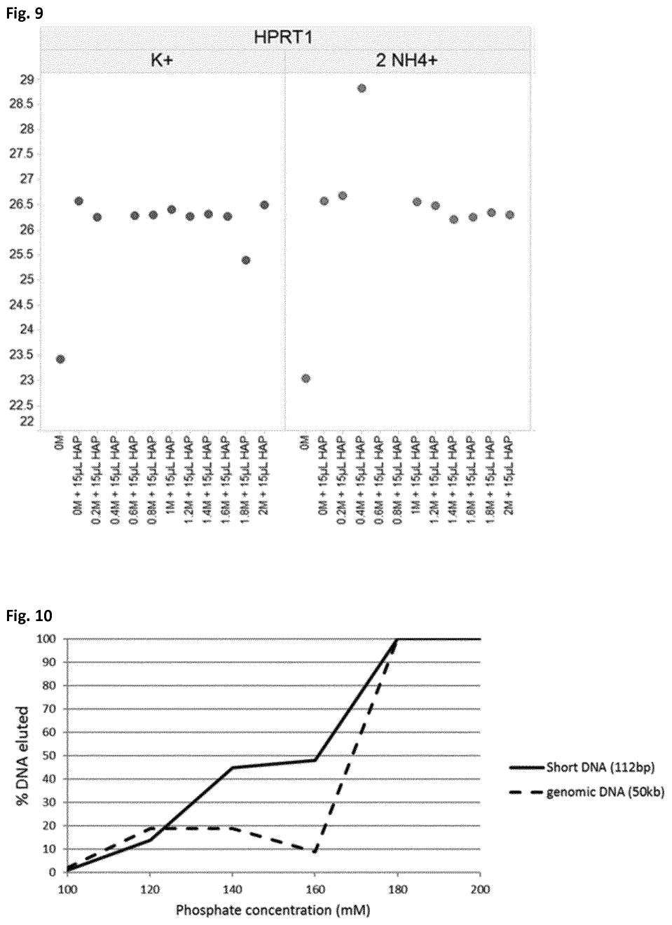

To ensure efficient binding of DNA to HAP, for example 1M NaCl and 100 mM MgCl₂ can be added in solid form to the plasma and HAP mixture. The high amount of these salts allows efficient binding of cfDNA to HAP. This is illustrated in FIG. 6 showing OD260 measurements of the unbound cfDNA fraction in the supernatants of the HAP-extracted plasma samples. Low detection of DNA represents a more efficient binding. From the FIG. 6, it can also be concluded that the contribution of the divalent ion (Mg2+) clearly benefits the binding efficiency. We have tested other mono- or divalent salts (FIGS. 7-9) or combinations thereof and concluded the best effects are obtained for Na⁺ and/or Mg²⁺ (FIGS. 7 and 8), possibly for Li+ (data not shown), and that, for example K⁺ or NH₄⁺; (FIG. 9) do not enhance DNA binding to HAP. After properly mixing the sample with HAP and salts of choice, the mixture is incubated at room temperature for one minute. The reaction is terminated by centrifugation of the sample at 3000 rpm for 30 seconds. The supernatant is subsequently removed from the HAP pellet on which the cfDNA is bound

The HAP pellet is then dissolved in a chose volume of a phosphate-containing buffer. In our example, the HAP pellet was dissolved in 1 mL of 0.5M KHPO₄ at pH 6.8, then incubated at room temperature for one minute. After this, HAP can be removed (e.g. via centrifugation or filtration) and the eluate containing cfDNA can be subjected to further analysis like e.g. in an automated workflow like the one described above.

FIG. 10 shows that the elution of DNA from the HAP matrix is correlated to the strand size of the DNA fragments. Longer DNA strands have a longer phosphate backbone and a stronger affinity for the HAP matrix. To ensure efficient elution of such strands, a higher phosphate concentration is required. The phosphate ions will compete with DNA for the calcium ion binding sites on the HAP matrix. In present experiments, we decided to use phosphate concentration of 0.5M, which provides a very fast and efficient DNA elution. Furthermore, the density of the eluted product very compatible downstream processing and with being transported along the fluidic path of the cartridge. For example, the sample density is of very high importance for the mixing efficiency with the chaotropic buffer used in Boom protocol, which is a feature that can be easily adapted within the scope of the present invention by the skilled person.

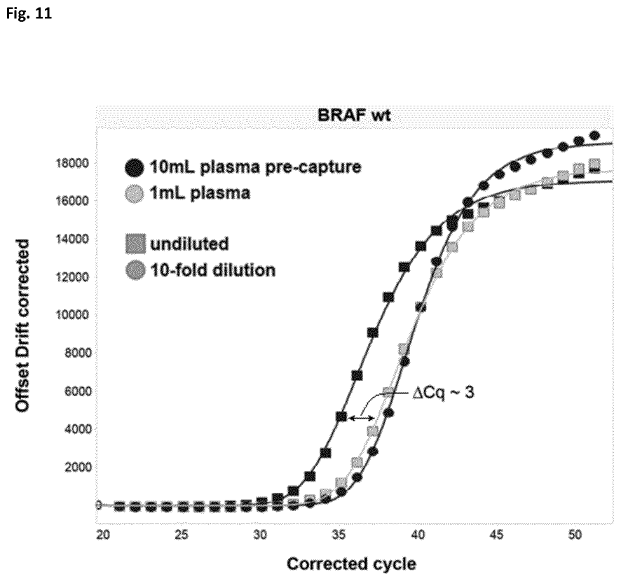

FIG. 11 illustrates the efficiency of the centrifugation-based HAP-extraction protocol. cfDNA from a 10 mL plasma sample was pre-captured and concentrated in 1 mL of 0.5M KHPO₄ at pH 6.8. The concentrated sample was then processed in the Idylla™ cartridge with silica-based extraction side by side with a regular 1 mL plasma sample. The eluted products were analysed by qPCR as described above. The qPCR curves provide relative quantification of the DNA concentration in the eluates. By definition, a 10-fold target increase should correspond with a Ct-shift of 3.3, which can be observed from present experiment when comparing the signal of the 10 mL sample with the signal of the 1 mL sample. This indicates that the extraction efficiency of the presented herein HAP-extraction protocol is ˜100%.

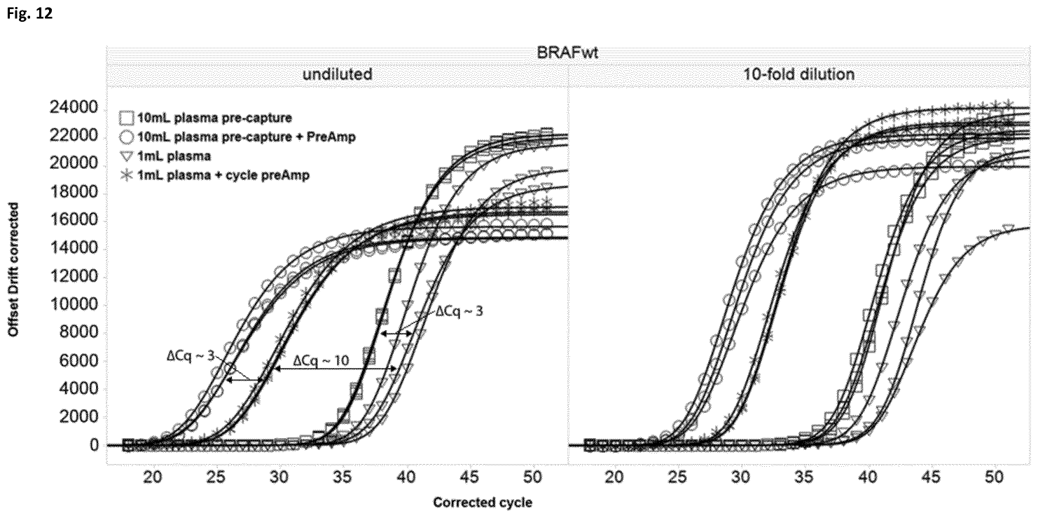

Finally, the results of the complete sequential workflow combining pre-amplification and HAP extraction are shown in FIG. 12. The results were obtained including HAP-based pre-capture of cfDNA from 10 mL of plasma followed by silica-based extraction and pre-amplification of the captured DNA as described above. This graph clearly a robust and significant increase of detected target copies in the downstream analysis. The sample volume and the amount of pre-amplification cycles can be easily scaled up or down but these proof-of-concept results clearly show that the present methodologies allow to maximize in an automated or semi-automated workflow the recuperation of low loads of cfDNA from liquid biopsies.

The present invention therefore shows a great potential for detecting low copy number nucleic acids in automated workflows where silica-based nucleic acid extraction is used, even from very diluted or large volume liquid samples. The provided herein pressure-controlled pre-amplification of silica-captured DNA can be used for upstream sample enrichment in a wide range of applications, essentially to increase the sensitivity of the downstream analysis. This could be next generation sequencing (NGS), or real-time PCR, where sample distribution over multiple reaction wells is often necessary. The rapid hydroxyapatite-based cfDNA extraction protocol, on the other hand, enables the processing of large volumes of bio-fluids, without losing extraction efficiency. The eluted product consists out of clean cfDNA dissolved in a 0.5M KHPO₄ buffer, and is compatible with most commercial downstream sample purification platforms. The embodiments of the invention have therefore the potential to be applied to and highly improve the sequential workflows and the sensitivity of many existing and future (semi-) automated molecular diagnostic platforms.

### DEFINITIONS

As used herein, the term “biological sample”, or simply “sample”, is intended to include a variety of biological sources that contain nucleic acid and/or cellular material, irrespective whether it is freshly obtained from an organism (i.e. fresh tissue sample) or preserved by any method known in the art (e.g. an frozen or an FFPE sample). Examples of biological samples include: cultures of cells such as mammalian cells but also of eukaryotic microorganisms, body fluids, body fluid precipitates, lavage specimen, fine needle aspirates, biopsy samples, tissue samples, cancer cells, other types of cells obtained from a patient, cells from a tissue or in vitro cultured cells from an individual being tested and/or treated for disease or infection, or forensic samples. Non-limiting examples of body fluid samples include whole blood, bone marrow, cerebrospinal fluid (CSF), peritoneal fluid, pleural fluid, lymph fluid, serum, plasma, urine, chyle, stool, ejaculate, sputum, nipple aspirate, saliva, swabs specimen, wash or lavage fluid and/or brush specimens.

The term “nucleic acid” and its equivalent “polynucleotide”, as used herein, refer to a polymer of ribonucleotides or deoxyribonucleotides bound together by phosphodiester linkages between the nucleotide monomers. (Deoxy)nucleotides are phosphorylated forms of (deoxy)nucleosides, which most commonly include adenosine, guanosine, cytidine, thymidine, or uridine. These nucleosides consist of a pentose sugar, being ribose or deoxyribose, and a nitrogenous base (“nucleobase”, or simply, “base”) being either adenine, guanine (that are purines), cytosine, thymine, or uracil (being pyrimidines). The sequence at which these bases (or their nucleosides, or the nucleotides of the latter) follow in a nucleic acid strand is termed “nucleic acid sequence” and is conventionally given in a so called 5′-end to 3′-end direction referring to chemical orientation of the nucleic acid stand. The “5′” originates from the reference to the 5′ carbon of the first (deoxy)ribose ring from which the reading of the nucleic acid sequence begins, and the “3′” originates from the 3′ carbon of the last (deoxy)ribose ring on which the reading of the nucleic acids sequence ends. A nucleic acid sequences can e.g. be ATATGCC, which is to be interpreted herein as referring to 5′-ATATGCC-3′ nucleic acid sequence. Under the same convention, the latter sequence will be complementary to the sequence 5′-GGCATAT-3′, or simply GGCATAT. Nucleic acids include but are not limited to DNA and RNA, including genomic DNA, mitochondrial or meDNA, cDNA, mRNA, rRNA, tRNA, hnRNA, microRNA, lncRNA, siRNA, and various modified versions thereof. Nucleic acids can most commonly be obtained from natural sources like biological samples obtained from different types of organisms. On the other hand, nucleic acids can also be synthesized, recombined, or otherwise produced in any of the known human-devised methods (e.g. PCR).

The term “quantitative PCR” or simply “qPCR” is herein given the definition of a laboratory technique based on the polymerase chain reaction (PCR), which is used to amplify and simultaneously detect or quantify a targeted DNA molecule. In contrast to standard PCR where the product of the reaction is detected at its end, i.e. after thermocycling has finished, the key feature of qPCR is that the DNA product is being detected during thermocycling as the reaction progresses in “real time”; hence, the alternative name of qPCR “real-time PCR”. There currently exist many different types of qPCRs. For example, when starting with a reverse transcription (RT) step, qPCR can be used to quantify numbers of messenger RNAs and is then called a reverse transcriptase qPCR or an RT-qPCR. As used herein the terms “quantitative PCR” or simply “qPCR” will be employed with preference over the term “real-time PCR” or “RT-PCR” in order to avoid confusion with reverse transcription PCR, also frequently abbreviated as RT-PCR. Most qPCRs use one of the two most common methods for detecting the product amplification in real-time: (a) intercalation of non-specific fluorescent dyes with any double-stranded DNA, or (2) sequence-specific DNA probes consisting of oligonucleotides that are labelled with a fluorescent reporter which permits detection only after hybridization of the probe with its complementary target sequence. The fluorescent signals generated during thermocycling are detected by an appropriate optical detection system and tracked from the moment they pass the background threshold till the reaction reaches plateau. The copy number of the target sequences can be estimated using either relative or absolute quantification strategy, typically by analysing the shape of the obtained amplification curve (standard curve strategy) or by determining when the signal rises above some threshold value (often called the Ct value, but sometimes also Cp value or Cq value). In relative quantification, the target nucleic acid levels estimated in a given sample using the Ct or standard curve analysis are expressed as relative to values obtained for the same target in another reference sample, for example, an untreated control sample. Conversely, in absolute quantification the qPCR signal is related to input copy number using a standard curve or can also be calculated according to a more recent digital PCR method. For the moment being, the first strategy is still more prevalent and bases the estimation of the target DNA amount by comparing the obtained values with a previously made standard curve. These and other qPCR quantification strategies are broadly known in the art and their calculation can differ in smaller or greater depending on a given application and a qPCR system.

As used herein, the term “means for performing quantitative PCR” shall be understood as minimum necessary arrangement of reagents and elements for performing a qPCR. They will usually include any reagents allowing detectable in real time PCR thermocycling of a nucleic acid template received from a source of nucleic acid. Such reagents include but depending on the type of qPCR are not limited to a PCR-grade polymerase, at least one primer set, a detectable dye or a probe, dNTPs, PCR buffer etc. Further, the “means for performing quantitative PCR” will usually also include any standard known in the art minimal assembly of parts, which usually includes but is not limited to the following: (1) a suitable compartment (further referred to as a “qPCR amplification chamber) where the real time-detectable thermocycling can take place. Such compartments can e.g. be formed by a chamber suitable for amplifying nucleic acids, i.e. made from appropriate material and providing for sufficient internal temperature regulation, and also comprising at least one wall allowing real-time detection of signals generated during such amplification, e.g. a wall transparent to light. Further, (2) means for varying temperature in this chamber, as broadly known from various existing thermocycling machines. Then, (3) means for detecting the signals generated during the qPCR thermocycling, like an optical detector coupled to a computer etc. In brief, such minimal assembly will normally include any known in the art system or systems capable of initiating and maintaining the thermocycling reaction in the thermocycling qPCR compartment for determined time, adjusting and regulating the temperature to ensure stable thermocycling conditions therein etc. Further, it will also include any appropriate detection device or devices, means for data processing (e.g. a computer), and output systems allowing to read and monitor the thermocycling of the qPCR reaction in real-time (usu. a computer screen displaying the reaction progress in an appropriate graphic user interface), as well as any software packages suitable for operating the machinery and/or displaying and possibly also aiding the interpretation of the obtained results.

As used herein, the term “cartridge” is to be understood as a self-contained assembly of chambers and/or channels, which is formed as a single object that can be transferred or moved as one fitting inside or outside of a larger instrument that is suitable for accepting or connecting to such cartridge. A cartridge and its instrument can be seen as forming an automated system, further referred to as an automated platform. Some parts contained in the cartridge may be firmly connected whereas others may be flexibly connected and movable with respect to other components of the cartridge. Analogously, as used herein the term “fluidic cartridge” shall be understood as a cartridge including at least one chamber or channel suitable for treating, processing, discharging, or analysing a fluid, preferably a liquid. An example of such cartridge is given in WO2007004103. Advantageously, a fluidic cartridge can be a microfluidic cartridge. In the context of fluidic cartridges the terms “downstream” and “upstream” can be defined as relating to the direction in which fluids flow in such cartridge. Namely, a section of a fluidic path in a cartridge from which a fluid flows towards a second section in the same cartridge is to be interpreted as positioned upstream of the latter. Analogously, the section to which a fluid arrives later is positioned downstream with respect to a section which said fluid passed earlier.

A cartridge is an example of an assembly that performs an “integrated workflow” of procedures that form part of a sample processing workflow, being the plurality of steps that lead to processing or modifying a sample with a particular purpose in mind. In this context, the term “integrated workflow” is to be understood as a series of processing steps that are performed in a largely automated manner, preferably was part of one fully automated (i.e. performed by an automated system, e.g. a robot or a similar machine or a series or a line of machines), or semi-automated (i.e. largely performed in an automated manner but requiring a minor manual or bench-top involvement from a user).

In general, as used herein the terms “fluidic” or sometimes “microfluidic” refers to systems and arrangements dealing with the behaviour, control, and manipulation of fluids that are geometrically constrained to a small, typically sub-millimetre-scale in at least one or two dimensions (e.g. width and height or a channel). Such small-volume fluids are moved, mixed, separated or otherwise processed at micro scale requiring small size and low energy consumption. Microfluidic systems include structures such as micro pneumatic systems (pressure sources, liquid pumps, micro valves, etc.) and microfluidic structures for the handling of micro, nano- and picoliter volumes (microfluidic channels, etc.). Exemplary fluidic systems were described in EP1896180, EP1904234, and EP2419705 and can accordingly be applied in certain embodiments of the presented herein invention.

In line with the above, the term “chamber” is to be understood as any functionally defined compartment of any geometrical shape within a fluidic or microfluidic assembly, defined by at least one wall and comprising the means necessary for performing the function which is attributed to this compartment. Along these lines, “amplification chamber” is to be understood as a compartment within a (micro)fluidic assembly, which suitable for performing and purposefully provided in said assembly in order to perform amplification of nucleic acids. Examples of an amplification chamber include a PCR chamber and a qPCR chamber. Similarly the terms “extraction chamber” or “nucleic acid extraction chamber”, alternatively “isolation chamber” or “nucleic acid isolation chamber”, alternatively “purification chamber” or “nucleic acid purification chamber” are to be understood as synonyms referring to a compartment within a fluidic or microfluidic assembly comprising the means to extract, isolate, or purify nucleic acid from a source of nucleic acid, possibly being a biological sample, and provide said nucleic acid in a form (e.g. aqueous solution) suitable for downstream analysis such as amplification and/or detection. A particular type of such chamber adapted for processing DNA shall be referred herein as “DNA extraction chamber” or “DNA isolation chamber” or “DNA purification chamber”, which should all be treated as synonyms. For the particular purposes of the present specification, unless specified otherwise, the terms referring to such “extraction/isolation/purification chamber” should be implicitly understood as comprising silica matrix suitable for extracting/isolating/purifying a nucleic acid in accordance with the principles of the Boom extraction method. Of note, the term “Boom method” is well known and clear in the art as referring to solid phase nucleic acid extraction strategy using silica, for reference cf e.g. U.S. Pat. No. 5,234,809 or EP0389063, and R Boom, C J Sol, M M Salimans, C L Jansen, P M Wertheim-van Dillen and J van der Noordaa; “Rapid and simple method for purification of nucleic acids.” J. Clin. Microbiol. March 1990 vol. 28 no. 3 495-503.

Lastly, as used herein the term “pre-amplification”, sometimes abbreviated in Figures to “pre-amp”, is to be construed broadly as referring to any nucleic acid amplification protocol that precedes another nucleic acid amplification protocol that is performed within an integrated workflow in a functionally defined amplification chamber. In particular contexts of the present specification, the term “pre-amplification” may refer to a pre-amplification protocol performed in an “extraction/isolation/purification chamber”.

1-15. (canceled)

16. A method of detecting a low copy number nucleic acid in an automated system, the method comprising:
contacting a biological sample with a hydroxyapatite (HAP) solid support to obtain a sample comprising a low copy number nucleic acid;
providing the sample comprising the low copy number nucleic acid to an extraction chamber of the automated system, said extraction chamber comprising silica surface for DNA adsorption;
adsorbing the nucleic acid to the silica surface; and
amplifying the nucleic acid within the automated system.

17. The method according to claim 16, wherein the contacting of the biological sample with the hydroxyapatite solid support is performed in the presence of Na⁺. Li⁺, or Mg²⁺ cations.

18. The method according to claim 16, wherein the sample comprising the low copy number nucleic acid is provided by eluting the nucleic acid captured to the hydroxyapatite solid support with a phosphate buffer, preferably comprising KHPO₄.

19. The method according to claim 16, wherein the step of amplifying is preceded by pre-amplifying the nucleic acid adsorbed to the silica surface, and optionally by eluting and transporting the pre-amplified nucleic acid to an amplification chamber of the automated system, said amplification chamber being fluidly connected with the extraction chamber.

20. The method according to claim 16, wherein the automated system comprises a cartridge adapted for the amplifying the nucleic acid.

21. The method according to claim 16, wherein the contacting of the biological sample with the HAP solid support is centrifugation-based.

22. The method according to claim 20, wherein the contacting of the biological sample with the HAP solid support is implemented as part of a fully automated workflow on the automated system, optionally wherein the contacting is implemented inside of the cartridge or via coupling a specific-volume-accommodating module to the cartridge.

23. The method according to claim 19, wherein the automated system is configured:
to position the pre-amplification reaction to only a portion of the silica surface occupying a zone of the extraction chamber adjacent to a heater, and
to maintain the remaining portion of the silica surface within the extraction chamber as substantially free of the pre-amplification reaction.

24. The method according to claim 19, comprising:
eluting the pre-amplified nucleic acid from the silica surface; and preferably
transporting the pre-amplified nucleic acid to the amplification chamber; and wherein the
amplifying comprises amplifying the pre-amplified nucleic acid in said amplification chamber.

25. The method according to claim 23, wherein the positioning is done by pressure control.

26. The method according to claim 19, wherein the pre-amplifying comprises symmetrical heat cycling between a top and a bottom temperature.

27. The method according to claim 23, wherein each of the top and the bottom temperature are held for at least 30 seconds, preferably for at least 45 second, most preferably for at least 1 minute.

28. The method according to claim 16, wherein the biological sample is a bodily fluid.

29. The method according to claim 28, wherein the bodily fluid is selected from plasma, serum, blood, urine, cerebrospinal fluid, bile, or saliva.
